(19) United States
(12) Patent Application Publication
(10) Pub. No.: US 2005/0084910 A1
(43) Pub. Date: Apr. 21, 2005

(54) Method for measuring FceRII/CD23 IgE receptor in human feces as an indicator of a TH2 predominant immune response involved in gastrointestinal illnesses

(76) Inventors: James Hunter Boone, Christiansburg, VA (US); David Maxwell Lyerly, Radford, VA (US); Tracy Dale Wilkins, Riner, VA (US)

Correspondence Address: SHOOK, HARDY & BACON LLP, 2555 GRAND BLVD, KANSAS CITY,, MO, 64108, US

(21) Appl. No.: 10/929,611

(22) Filed: Aug. 30, 2004

Related U.S. Application Data
(60) Provisional application No. 60/498,772, filed on Aug. 29, 2003.

(51) Int. Cl. G01N 33/53

(52) U.S. Cl. 435/7.1

(57) ABSTRACT

A method and apparatus for measuring FcεRII/CD23 IgE receptor in human feces or other biological specimens as an indicator of a TH2 immune response indicative of gastrointestinal illnesses such as ulcerative colitis and food allergy is described. The apparatus consists of either a qualitative or quantitative enzyme-linked immunoassay or other immunoassay that utilizes antibodies specific to human FcεRII/CD23 IgE receptor for the measurement of endogenous FcεRII/CD23 IgE receptor in human feces. The method and apparatus can be used by healthcare providers as an aid for determining gastrointestinal illnesses that are predominantly a TH2 predominant immune response such as ulcerative colitis, large bowel Crohn's disease (IC-like Crohn's disease), allergic colitis (food allergy) and parasitic infections.

### CROSS-REFERENCE TO RELATED APPLICATIONS

This application claims the benefit of priority to U.S. Provisional Application No. 60/498,772 filed on Aug. 29, 2003.

### STATEMENT REGARDING FEDERALLY SPONSORED RESEARCH OR DEVELOPMENT

Not applicable.

### BACKGROUND OF THE INVENTION

The FcεRII/CD23 IgE receptor is a membrane protein expressed by a wide range of cells including B lymphocytes, T lymphocytes, monocytes and eosinophils. This low-affinity IgE receptor binds the IgE immunoglobulin and signals the release of histamine as part of the TH2 immune response and regulates the production of IgE. The FcεRII/CD23 IgE receptor is proteolytically cleaved from the membrane surface, releasing a stable soluble 25 kD fragment. The soluble FcεRII/CD23 acts as a cytokine to stimulate the production of IL-5 for the recruitment of eosinophils and IL-6 for the differentiation of B lymphocytes. In in vitro experiments, soluble FcεRII/CD23 has been shown to interact with P2 integrin CD11b to stimulate proinflammatory TNF-α production by monocytes (macrophages). The FcεRII/CD23 glycoprotein is expressed at higher levels in activated eosinophils, increasing the levels of FcεRII/CD23 during cell infiltration in diseased tissue.

Currently, there are serum-based immunoassays for the detection of soluble serum FcεRII/CD23 IgE receptor as an indicator of rheumatoid arthritis and chronic lymphocytic leukemia (Bindazyme™ sCD23, The Binding Site, U.K.). However, this method does not determine the presence of elevated fecal FcεRII/CD23 IgE receptor as a specific marker for a TH2 immune response indicating gastrointestinal illnesses such as allergic colitis (food allergy), ulcerative colitis and large bowel Crohn's disease, and parasitic infections. There remains a need for a method utilizing antibodies specific for FcεRII/CD23 IgE receptor for measuring elevated fecal FcεRII/CD23 IgE receptor as an indicator of allergic colitis and other eosinophilic gastrointestinal illnesses such as food allergy that involve a TH2 immune response.

### SUMMARY OF THE INVENTION

The present invention relates to methods for aiding in the determination of a TH2 immune response by determining the presence of elevated FcεRII/CD23 IgE receptor in human feces as a marker for TH2 predominant gastrointestinal illnesses. In addition, the presence of elevated fecal FcεRII/CD23 IgE receptor can be used to determine gastrointestinal illnesses such as allergic colitis, ulcerative colitis, large bowel Crohn's disease (UC-like Crohn's disease) and parasitic infections that involve a TH2 immune response. The measurement of elevated fecal FcεRII/CD23 offers a diagnostic aid for the determination of TH2 predominant gastrointestinal illnesses for optimizing medical treatment.

### BRIEF DESCRIPTION OF THE DRAWING

FIG. 1 is a graph generated using optical density values in accordance with an embodiment of the present invention.

### DETAILED DESCRIPTION OF THE INVENTION

The fecal and serum FcεRII/CD23 IgE receptor specific immunoassays can be used to determine a gastrointestinal illnesses with a TH2 predominant immune response such as in the case of inflammatory bowel disease and allergic colitis for determining the optimal treatment by measuring the presence of elevated fecal FcεRII/CD23 IgE receptor (human feces and mucosal secretions). The detection of elevated fecal FcεRII/CD23 IgE receptor can also be useful for indicating other TH2 predominant gastrointestinal illnesses such as allergic colitis in cases of food allergy and other eosinophilic gastrointestinal illnesses.

A qualitative immunoassay such as a lateral flow and flow-through tests that utilize antibodies to protein fragments of human FcεRII/CD23 IgE receptor for detecting elevated FcεRII/CD23 IgE receptor in human feces, saliva or mucosal secretions can indicate the absence or presence of a TH2 immune response for predominant diseases such as allergic colitis, ulcerative colitis, large bowel Crohn's disease and parasitic infections.

In the qualitative assay, human feces is diluted and 100 μl of diluted specimen to an individual well of a microassay plate coated with antibodies, proteins or polypeptide fragments specific to FcεRII/CD23 IgE receptor. If endogenous fecal FcεRII/CD23 IgE receptor is present, it will bind to the capture antibody, proteins or polypeptide fragments during an incubation step for 1 to 2 hours. Following incubation, anti-human specific antibodies conjugated to an enzyme, anti-IgG-Horse-radish peroxidase (HRP) or streptavidin-HRP Conjugate is added and allowed to bind with antibodies to captured fecal FcεRII/CD23 IgE receptor. The test wells are incubated to allow binding of the conjugate. Unbound conjugate is then washed from the well and substrate (tetramethylbenzidene/peroxide) is added for color development. Following the substrate incubation, low pH stop solution (sulfuric acid) is added to stop the reaction and the optical density (OD) is obtained spectrophotometrically at 450 nm.

In our clinical study, we screened 49 healthy subjects (no known acute infection or chronic intestinal disorders) to determine the baseline readings for fecal FcεRII/CD23. A total of 8 (16.3%) of the 49 subjects had detectable FcεRII/CD23, indicating a TH2 immune response. Measurable FcεRII/CD23 was then evaluated in 79 pediatric subjects with inflammatory bowel disease (IBD) and 6 irritable bowel syndrome (IBS) patients. Of the 79 IBD patients, a total of 25 subjects had detectable levels of fecal FcεRII/CD23 (8 Crohn's disease and 17 ulcerative colitis). There was a 2:1 ratio of UC to CD, indicating a higher frequency of a TH2 immune response in UC than in CD. A single subject with IBS had a detectable level of fecal FcεRII/CD23. A summary of results is shown below in Table 1 and subject data are shown in Tables 2 through 4.

TABLE 1
Detection of FcεRII/CD23 in Fecal Specimens from subjects with
Crohn's disease, ulcerative colitis, IBS and in healthy persons.

| Total | | Fecal FcεRII/CD23 | Fecal FcεRII/CD23 |
|---|---|---|---|
| Assessments N = 134 | Total | Positive | Negative |
| Total IBD (Crohn's | 79 | 31.7% (25) | 68.3% (54) |
| disease and | | | |
| ulcerative colitis) | | | |
| Total Crohn's Disease | 38 | 21.1% (8) | 78.9% (30) |
| Total Ulcerative | 41 | 41.5% (17) | 58.5% (24) |
| Colitis | | | |
| Total Active IBS | 6 | 16.7% (1) | 83.3% (5) |
| Healthy Persons | 49 | 16.3% (8) | 83.7% (41) |

TABLE 2
FcεRII/CD23 results for healthy subjects

| | | | FcεRII/CD23 | |
|---|---|---|---|---|
| | | Presence of | Test | |
| Subject | | intestinal | Absorbance | |
| ID | Disease | inflammation | value (OD_{450 nm}) | Interpretation |
| H1 | Healthy | None | 0.111 | NEGATIVE |
| H2 | Healthy | None | 0.115 | NEGATIVE |
| H3 | Healthy | None | 0.144 | NEGATIVE |
| H4 | Healthy | None | 0.095 | NEGATIVE |
| H5 | Healthy | None | 0.113 | NEGATIVE |
| H6 | Healthy | None | 0.241 | POSITIVE |
| H7 | Healthy | None | 0.130 | NEGATIVE |
| H8 | Healthy | None | 0.182 | POSITIVE |
| H9 | Healthy | None | 0.113 | NEGATIVE |
| H10 | Healthy | None | 0.103 | NEGATIVE |
| H11 | Healthy | None | 0.128 | NEGATIVE |
| H12 | Healthy | None | 0.119 | NEGATIVE |
| H13 | Healthy | None | 0.099 | NEGATIVE |
| H14 | Healthy | None | 0.099 | NEGATIVE |
| H15 | Healthy | None | 0.119 | NEGATIVE |
| H16 | Healthy | None | 0.112 | NEGATIVE |
| H17 | Healthy | None | 0.104 | NEGATIVE |
| H18 | Healthy | None | 0.107 | NEGATIVE |
| H19 | Healthy | None | 0.092 | NEGATIVE |
| H20 | Healthy | None | 0.096 | NEGATIVE |
| H21 | Healthy | None | 0.095 | NEGATIVE |
| H22 | Healthy | None | 0.101 | NEGATIVE |
| H23 | Healthy | None | 0.096 | NEGATIVE |
| H24 | Healthy | None | 0.124 | NEGATIVE |
| H25 | Healthy | None | 0.078 | NEGATIVE |
| H26 | Healthy | None | 0.102 | NEGATIVE |
| H27 | Healthy | None | 0.114 | NEGATIVE |
| H28 | Healthy | None | 0.141 | NEGATIVE |
| H29 | Healthy | None | 0.097 | NEGATIVE |
| H30 | Healthy | None | 0.107 | NEGATIVE |
| H31 | Healthy | None | 0.091 | NEGATIVE |
| H32 | Healthy | None | 0.095 | NEGATIVE |
| H33 | Healthy | None | 0.091 | NEGATIVE |
| H34 | Healthy | None | 0.225 | POSITIVE |
| H35 | Healthy | None | 0.115 | NEGATIVE |
| H36 | Healthy | None | 0.222 | POSITIVE |
| H37 | Healthy | None | 0.227 | POSITIVE |
| H38 | Healthy | None | 0.147 | NEGATIVE |
| H39 | Healthy | None | 0.181 | POSITIVE |
| H40 | Healthy | None | 0.188 | POSITIVE |
| H41 | Healthy | None | 0.103 | NEGATIVE |
| H42 | Healthy | None | 0.096 | NEGATIVE |
| H43 | Healthy | None | 0.137 | NEGATIVE |
| H44 | Healthy | None | 0.129 | NEGATIVE |
| H45 | Healthy | None | 0.167 | POSITIVE |
| H46 | Healthy | None | 0.096 | NEGATIVE |
| H47 | Healthy | None | 0.111 | NEGATIVE |
| H48 | Healthy | None | 0.124 | NEGATIVE |
| H49 | Healthy | None | 0.126 | NEGATIVE |

Cutoff = ≧0.157

TABLE 3
FcεRII/CD23 results for IBD subjects

| | | | FcεRII/CD23 | |
|---|---|---|---|---|
| | | | Test | |
| Subject | | Disease | Absorbance | |
| ID | Disease | Activity | value (OD_{450 nm}) | Interpretation |
| IBD1 | CD | ACTIVE | 0.123 | NEGATIVE |
| IBD2 | CD | ACTIVE | 0.138 | NEGATIVE |
| IBD3 | CD | ACTIVE | 0.116 | NEGATIVE |
| IBD4 | UC | INACTIVE | 0.116 | NEGATIVE |
| IBD5 | CD | ACTIVE | 0.153 | NEGATIVE |
| IBD6 | UC | INACTIVE | 0.121 | NEGATIVE |
| IBD7 | UC | ACTIVE | 0.169 | POSITIVE |
| IBD8 | CD | ACTIVE | 0.305 | POSITIVE |
| IBD9 | UC | INACTIVE | 0.116 | NEGATIVE |
| IBD10 | CD | INACTIVE | 0.123 | NEGATIVE |
| IBD11 | UC | ACTIVE | 0.180 | POSITIVE |
| IBD12 | CD | ACTIVE | 0.111 | NEGATIVE |
| IBD13 | UC | ACTIVE | 0.111 | NEGATIVE |
| IBD14 | UC | ACTIVE | 0.103 | NEGATIVE |
| IBD15 | UC | ACTIVE | 0.158 | POSITIVE |
| IBD16 | UC | INACTIVE | 0.157 | POSITIVE |
| IBD17 | UC | ACTIVE | 0.138 | NEGATIVE |
| IBD18 | UC | ACTIVE | 0.131 | NEGATIVE |
| IBD19 | CD | ACTIVE | 0.107 | NEGATIVE |
| IBD20 | UC | ACTIVE | 0.235 | POSITIVE |
| IBD21 | UC | ACTIVE | 0.186 | POSITIVE |
| IBD22 | CD | ACTIVE | 0.116 | NEGATIVE |
| IBD23 | UC | ACTIVE | 0.273 | POSITIVE |
| IBD24 | CD | ACTIVE | 0.098 | NEGATIVE |
| IBD25 | CD | INACTIVE | 0.115 | NEGATIVE |
| IBD26 | UC | ACTIVE | 0.129 | NEGATIVE |
| IBD27 | CD | INACTIVE | 0.128 | NEGATIVE |
| IBD28 | CD | ACTIVE | 0.150 | NEGATIVE |
| IBD29 | UC | ACTIVE | 0.129 | NEGATIVE |
| IBD30 | UC | ACTIVE | 0.119 | NEGATIVE |
| IBD31 | CD | ACTIVE | 0.171 | POSITIVE |
| IBD32 | CD | ACTIVE | 0.313 | POSITIVE |
| IBD33 | CD | ACTIVE | 0.116 | NEGATIVE |
| IBD34 | CD | ACTIVE | 0.152 | NEGATIVE |
| IBD35 | UC | ACTIVE | 0.335 | POSITIVE |
| IBD36 | CD | ACTIVE | 0.123 | NEGATIVE |
| IBD37 | UC | ACTIVE | 0.129 | NEGATIVE |
| IBD38 | CD | ACTIVE | 0.108 | NEGATIVE |
| IBD39 | CD | ACTIVE | 0.109 | NEGATIVE |
| IBD40 | UC | ACTIVE | 0.181 | POSITIVE |
| IBD41 | UC | ACTIVE | 0.125 | NEGATIVE |
| IBD42 | UC | ACTIVE | 0.160 | POSITIVE |
| IBD43 | CD | ACTIVE | 0.116 | NEGATIVE |
| IBD44 | UC | INACTIVE | 0.112 | NEGATIVE |
| IBD45 | CD | ACTIVE | 0.099 | NEGATIVE |
| IBD46 | CD | ACTIVE | 0.367 | POSITIVE |
| IBD47 | UC | ACTIVE | 0.101 | NEGATIVE |
| IBD48 | CD | ACTIVE | 0.111 | NEGATIVE |
| IBD49 | CD | ACTIVE | 0.131 | NEGATIVE |
| IBD50 | CD | ACTIVE | 0.173 | POSITIVE |
| IBD51 | UC | ACTIVE | 0.194 | POSITIVE |
| IBD52 | UC | INACTIVE | 0.233 | POSITIVE |
| IBD53 | UC | ACTIVE | 0.118 | NEGATIVE |
| IBD54 | CD | ACTIVE | 0.120 | NEGATIVE |
| IBD55 | CD | ACTIVE | 0.137 | NEGATIVE |
| IBD56 | CD | INACTIVE | 0.129 | NEGATIVE |
| IBD57 | UC | ACTIVE | 0.191 | POSITIVE |
| IBD58 | UC | ACTIVE | 0.281 | POSITIVE |
| IBD59 | UC | INACTIVE | 0.205 | POSITIVE |
| IBD60 | CD | ACTIVE | 0.191 | POSITIVE |
| IBD61 | UC | ACTIVE | 0.122 | NEGATIVE |
| IBD62 | UC | INACTIVE | 0.136 | NEGATIVE |
| IBD63 | UC | ACTIVE | 0.347 | POSITIVE |
| IBD64 | CD | ACTIVE | 0.123 | NEGATIVE |
| IBD65 | CD | INACTIVE | 0.124 | NEGATIVE |
| IBD66 | UC | ACTIVE | 0.126 | NEGATIVE |
| IBD67 | UC | INACTIVE | 0.143 | NEGATIVE |
| IBD68 | UC | INACTIVE | 0.134 | NEGATIVE |
| IBD69 | UC | ACTIVE | 0.146 | NEGATIVE |
| IBD70 | CD | ACTIVE | 0.186 | POSITIVE |
| IBD71 | UC | ACTIVE | 0.193 | POSITIVE |
| IBD72 | CD | INACTIVE | 0.113 | NEGATIVE |
| IBD73 | UC | ACTIVE | 0.130 | NEGATIVE |
| IBD74 | CD | ACTIVE | 0.130 | NEGATIVE |
| IBD75 | CD | ACTIVE | 0.132 | NEGATIVE |
| IBD76 | UC | INACTIVE | 0.137 | NEGATIVE |
| IBD77 | CD | INACTIVE | 0.262 | POSITIVE |
| IBD78 | CD | ACTIVE | 0.115 | NEGATIVE |
| IBD79 | UC | INACTIVE | 0.145 | NEGATIVE |

Cutoff = ≧0.157

TABLE 4
FcεRII/CD23 results for IBS subjects

| | | | FcεRII/CD23 | |
|---|---|---|---|---|
| | | | Test | |
| Subject | | Disease | Absorbance | |
| ID | Disease | Activity | value (OD_{450 nm}) | Interpretation |
| IBS1 | IBS | SYMTOMATIC | 0.116 | NEGATIVE |
| IBS2 | IBS | SYMTOMATIC | 0.143 | NEGATIVE |
| IBS3 | IBS | SYMTOMATIC | 0.115 | NEGATIVE |
| IBS4 | IBS | SYMTOMATIC | 0.111 | NEGATIVE |
| IBS5 | IBS | SYMTOMATIC | 0.108 | NEGATIVE |
| IBS6 | IBS | SYMTOMATIC | 0.169 | POSITIVE |

Cutoff = ≧0.157

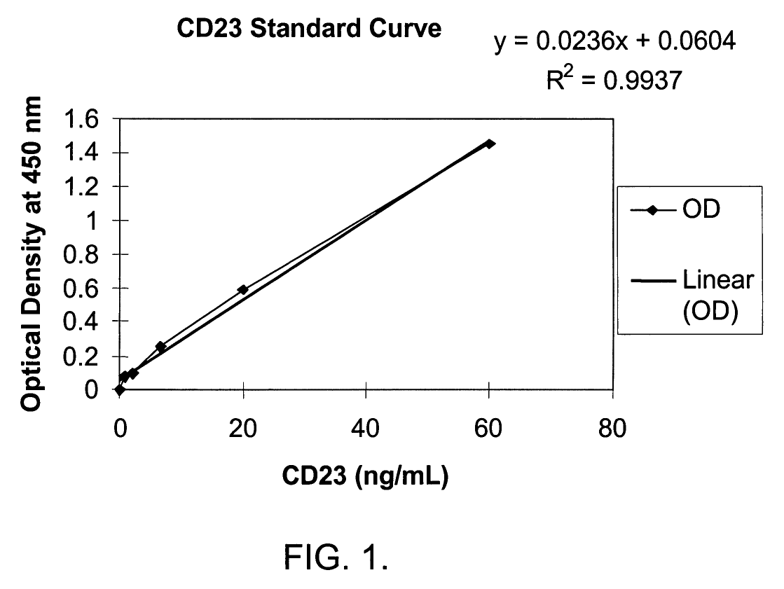

A standard curve was generated using the FcεRII/CD23 ELISA test and purified human FcεRII/CD23 for a quantitative assay. The curve shows linearity with an R² value of 0.99. The optical densities (OD) for each FcεRII/CD23 concentration and a graph generated using the OD values are shown below in Table 5 and FIG. 1. Using the FcεRII/CD23 standard curve, fecal specimens of healthy subjects were analyzed quantitatively for levels of fecal FcεRII/CD23. The mean±SD for fecal FcεRII/CD23 in healthy subjects was 6.8±9.5 ng/mL. The FcεRII/CD23 levels for each healthy subject are listed below in Table 6. A second test group, 3 adult subjects with UC and 3 adult subjects with CD were monitored for detectable levels of fecal FcεRII/CD23 over a range of 6 months with an average of 6 specimens (1 per month). The mean±SD for the subjects with IBD was 67.3±43.5 ng/mL. The mean level for healthy subjects was statistically different from the level determined in the subjects with IBD (Two-tailed Student T-Test; p<0.005). A majority of these subjects continued to show elevated levels for the 6-month period. The quantitative results for the IBD subjects are shown in Table 7.

TABLE 5
Standard curves generated using Purified Human FcεRII/CD23

| Purified | |
|---|---|
| FcεRII/CD23 | Mean |
| (ng/mL) | OD |
| 60 | 1.453 |
| 20 | 0.591 |
| 6.7 | 0.260 |
| 2.2 | 0.101 |
| 0.7 | 0.074 |

TABLE 6
FcεRII/CD23 levels for healthy subjects

| | Fecal |
|---|---|
| Subject | FcεRII/CD23 |
| Code | (ng/mL) |
| 001 | 5.6 |
| 003 | 7.5 |
| 007 | 3.6 |
| 008 | 5.3 |
| 011 | 12.8 |
| 012 | 3.3 |
| 014 | 4.7 |
| 015 | 10.8 |
| 016 | 16.0 |
| 017 | 9.3 |
| 020 | 7.2 |
| 021 | 0 |
| 025 | 14.4 |
| 026 | 0 |
| 033 | 0 |
| 035 | 4.5 |
| 037 | 0 |
| 039 | 6.4 |
| 042 | 7.0 |
| 043 | 16.4 |
| 045 | 57.9 |
| 046 | 4.2 |
| 047 | 0 |
| 049 | 1.6 |
| 050 | 31.7 |
| 052 | 0 |
| 055 | 0 |
| 056 | 0 |
| 064 | 5.0 |
| 065 | 6.4 |
| 066 | 12.5 |
| 068 | 6.8 |
| 070 | 3.2 |
| 071 | 0 |
| 072 | 5.3 |
| 073 | 3.6 |
| 077 | 0 |
| 078 | 5.5 |
| 080 | 0 |
| 081 | 19.0 |
| 082 | 5.3 |
| 083 | 4.1 |
| 084 | 0 |
| 086 | 0 |
| 087 | 3.3 |
| 088 | 0 |
| 090 | 5.0 |
| 094 | 6.2 |
| 096 | 4.4 |
| 097 | 3.6 |
| 099 | 0 |
| 112 | 17.9 |
| 113 | 20.6 |
| 114 | 0 |
| 120 | 7.8 |
| Mean | 6.8 ng/ml |
| Std. | 9.5 |

Std. = Standard deviation

TABLE 7
FcεRII/CD23 levels for IBD subjects over time

| | | | Fecal | Interpretation |
|---|---|---|---|---|
| Subject | | | FcεRII/CD23 | Level ≧ 25.8* = |
| ID | Disease | Sample date | level (ng/mL) | Elevated |
| 1CDF | CD | August 03 | 36.9 | ELEVATED |
| | | September 03 | 141.3 | ELEVATED |
| | | October 03 | 77.2 | ELEVATED |
| | | December 03 | 158.6 | ELEVATED |
| | | January 04 | 66.3 | ELEVATED |
| 1UCF | UC | August 03 | 16.4 | BASELINE |
| | | September 03 | 54.4 | ELEVATED |
| | | October 03 | 14.0 | BASELINE |
| | | November 03 | 136.9 | ELEVATED |
| | | December 03 | 142.6 | ELEVATED |
| | | January 04 | 16.4 | BASELINE |
| 2UCM | UC | August 03 | 34.5 | ELEVATED |
| | | September 03 | 123.8 | ELEVATED |
| 2UCM | UC | October 03 | 80.2 | ELEVATED |
| | | November 03 | 21.5 | BASELINE |
| | | December 03 | 57.4 | ELEVATED |
| 2CDF | CD | September 03 | 10.2 | BASELINE |
| | | October 03 | 22.6 | BASELINE |
| | | November 03 | 87.3 | ELEVATED |
| | | December 03 | 79.6 | ELEVATED |
| | | January 04 | 21.5 | ELEVATED |
| ACRM1 | CD | November 03 | 39.9 | ELEVATED |
| | | December 03 | 60.9 | ELEVATED |
| | | December 03 | 107.8 | ELEVATED |
| | | December 03 | 101.9 | ELEVATED |
| UCM4 | UC | November 03 | 30.7 | ELEVATED |
| | | November 03 | 65.4 | ELEVATED |
| | | December 03 | 67.2 | ELEVATED |
| | | January 04 | 77.8 | ELEVATED |
| | | Mean | 67.3 | |
| | | Std. | 43.5 | |

*Cut-off for elevated FcεRII/CD23 is the mean of healthy subjects plus 2 Std. (6.8 + 2(9.5) = 25.8).
Std. = Standard deviation

From the foregoing, it will be seen that this invention is well adapted to attain all the ends and objects hereinabove set forth, together with other advantages which are obvious and which are inherent to the method.

It will be understood that certain features and subcombinations are of utility and may be employed without reference to other features and subcombinations. This is contemplated by and is within the scope of the claims.

1. A method for detecting amounts of FcεRII/CD23 IgE receptor in a fecal sample, the method comprising:
obtaining a fecal sample from a human;
determining the presence of FcεRII/CD23 IgE receptor in the fecal sample.

2. The method of claim 1, wherein the presence of FcεRII/CD23 IgE receptor is determined using one of an enzyme linked immunoassay, flow-through membrane test and lateral flow membrane test.

3. The method of claim 2, further comprising:
determining whether the FcεRII/CD23 IgE receptor is elevated.

4. The method of claim 3, wherein if the FcεRII/CD23 IgE receptor is elevated, determining the presence of a TH2 immune response in the human.

5. The method of claim 4, wherein said TH2 immune response is indicative of allergic colitis.

6. The method of claim 4, wherein said TH2 immune response is indicative of TH2 predominant ulcerative colitis subgroup.

7. The method of claim 4, wherein said TH2 immune response is indicative of TH2 predominant Crohn's Disease subgroup.

8. The method of claim 4, wherein said TH2 immune response is indicative of a parasitic infection.

9. The method of claim 4, wherein said TH2 immune response is indicative of eosinophilic gastrointestinal illness.

10. The method of claim 1, wherein the FcεRII/CD23 IgE receptor comprises total FcεRII/CD23 IgE receptor in the fecal sample.

11. A method for detecting the presence of a TH2 response in a patient, the method comprising:
obtaining a fecal sample from a patient, wherein said fecal sample comprises FcεRII/CD23 IgE receptor;
contacting the fecal sample with antibodies specific to the FcεRII/CD23 IgE receptor;
detecting in the sample an amount of FcεRII/CD23 IgE receptor that binds to the one of antibodies and protein fragments specific to the FcεRII/CD23 IgE receptor; and
comparing the amount of bound receptor to a pre-determined cut-off value and therefrom determining the presence of a TH2 immune response in the patient.

12. The method of claim 11, wherein said TH2 immune response is indicative of allergic colitis.

13. The method of claim 11, wherein said TH2 immune response is indicative of a TH2 predominant ulcerative colitis subgroup.

14. The method of claim 11, wherein said TH2 immune response is indicative of a TH2 predominant Crohn's Disease subgroup.

15. The method of claim 11, wherein said TH2 immune response is indicative of a parasitic infection.

16. The method in claim 11, wherein the presence of fecal FcεRII/CD23 IgE receptor within a single patient is used as an aid in determining the optimal treatment and TH2 immune specific therapy such as anti-CD23 antibody infusions.

17. A assay for determining the amount of FcεRII/CD23 IgE receptor in human feces, the method comprising:
obtaining a sample of one of human feces, saliva and mucosal secretions;
diluting the sample;
contacting the sample with antibodies or proteins specific to FcεRII/CD23 IgE receptor and allowing the FcεRII/CD23 IgE receptors in the sample to bind to the antibodies or proteins to create a bound sample; contacting the bound sample with a conjugate to create a readable sample; and
determining the optical density of the readable sample at 450 nm to determine the level of FcεRII/CD23 IgE receptor.

18. The assay of claim 17, further comprising:
comparing the optical density of the readable sample to a predetermined cut-off value.

19. The assay of claim 18, wherein if the optical density of the readable sample is above a cut-off value, determining the presence of a TH2 immune response in the patient.

20. A kit for diagnosing a TH2 immune response in a person by testing a fecal sample from a person to be diagnosed, the kit comprising:
one or more microassay plates, each the plate containing antibodies or proteins specific to FcεRII/CD23 IgE receptor;
anti-human specific antibodies conjugated to an enzyme; and
enzyme substrate for color development.

21. The kit as recited in claim 21, further comprising a stop solution for quenching the reaction.
